(19) United States
(12) Patent Application Publication
(10) Pub. No.: US 2014/0012314 A1
(43) Pub. Date: Jan. 9, 2014

(54) CUPPED FORCEPS

(71) Applicant: InVivo Therapeutics Corporation, Cambridge, MA (US)

(72) Inventors: Haining William Dai, Rockville, MD (US); Brian Hess, Charlestown, MA (US); Francis M. Reynolds, Lafayette Hill, PA (US); David Kosh, Norton, MA (US); Fioleda Prifti, Paxton, MA (US)

(73) Assignee: InVivo Therapeutics Corporation, Cambridge, MA (US)

(21) Appl. No.: 13/930,829

(22) Filed: Jun. 28, 2013

Related U.S. Application Data
(60) Provisional application No. 61/666,317, filed on Jun. 29, 2012.

(51) Int. Cl. A61B 17/28

(52) U.S. Cl. CPC A61B 17/2804
USPC 606/207

(57) ABSTRACT

Cupped forceps are provided. Methods of using a cupped forceps are provided.

### CROSS REFERENCE TO RELATED APPLICATIONS

This application claims the benefit of U.S. provisional application 61/666,317, which was filed Jun. 29, 2012 and is incorporated herein by reference as if fully set forth.

### FIELD

The disclosure relates to a cupped forceps designed for scaffold handling and transport.

### BACKGROUND

Forceps are handheld, hinged instruments used for grasping and holding objects. A typical forceps is v-shaped, which applies point or edge forces to the object. Forceps are often used in the medical field.

### SUMMARY

In an aspect, the invention relates to a cupped forceps. The cupped forceps includes a first shaft having a first proximal end and a first distal end, and a second shaft having a second proximal end and a second distal end. The first distal end of the first shaft is connected to the second distal end of the second shaft. A first cupped tip having a first concavity between a first tip end and a second tip end is connected at the first proximal end of the first shaft. A second cupped tip having a second concavity between a third tip end and a fourth tip end is connected at the second proximal end of the second shaft. The first cupped tip concavity faces the second cupped tip concavity. The first shaft and the second shaft are pivotable towards each other about a pivot axis to a closed position. The first tip end meets the third tip end and the second tip end is advanced towards the fourth tip end in the closed position to form a closed unit. When closed the cupped tips may form a 270° inner circumference.

In an aspect, the invention relates to a cupped forceps. The cupped forceps includes a first shaft having a first proximal end and a first distal end and a second shaft having a second proximal end and a second distal end. The first distal end of the first shaft is connected to the second distal end of the second shaft. The cupped forceps includes a first branch having a first branch proximal end and a first branch distal end. The first branch distal end is connected to the first proximal end of the first shaft. The first branch proximal end includes a first prong and a second prong. The cupped forceps includes a second branch having a second branch proximal end and a second branch distal end. The second branch distal end is connected to the second proximal end of the second shaft. The second branch proximal end includes a third prong and a fourth prong. The cupped forceps includes a first cupped tip having a first concavity between a first tip end and a second tip end, the first tip end connected at the first prong. The cupped forceps includes a second cupped tip having a second concavity between a third tip end and a fourth tip end, the third tip end connected at the third prong. The cupped forceps includes a third cupped tip having a third concavity between a fifth tip end and a sixth tip end, the fifth tip end connected at the second prong. The cupped forceps includes a fourth cupped tip having a fourth concavity between a seventh tip end and an eighth tip end, the seventh tip end connected at the fourth prong. The first cupped tip concavity faces the second cupped tip concavity and the third cupped tip concavity faces the fourth cupped tip concavity. The first shaft and the second shaft are pivotable towards each other about a pivot axis to a closed position. The first tip end is configured to meet the third tip end. The fifth tip end is configured to meet the seventh tip end. The second tip end and the fourth tip end are configured to be advanced towards each other to form a closed unit in the closed position. The sixth tip end and the eighth tip end are configured to be advanced towards each other to form the closed unit in the closed position.

In an aspect, the invention relates to a method of handling an object. The method includes enclosing the object in a cupped forceps. The cupped forceps includes a first shaft having a first proximal end and a first distal end, and a second shaft having a second proximal end and a second distal end. The first distal end of the first shaft is connected to the second distal end of the second shaft. A first cupped tip having a first concavity between a first tip end and a second tip end is connected at the first proximal end of the first shaft. A second cupped tip having a second concavity between a third tip end and a fourth tip end is connected at the second proximal end of the second shaft. The first cupped tip concavity faces the second cupped tip concavity. The first shaft and the second shaft are pivotable towards each other about a pivot axis to a closed position. The first tip end meets the third tip end and the second tip end is advanced towards the fourth tip end in the closed position to form a closed unit.

### BRIEF DESCRIPTION OF THE DRAWINGS

The following detailed description of the embodiments will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 illustrates an embodiment of a cupped forceps in an open position.

FIG. 2A illustrates a closed unit of a cupped forceps.

FIG. 2B illustrates a side view of a cupped forceps.

FIGS. 3A-3B illustrate examples of cupped forceps.

FIGS. 4A-4B illustrate examples of cupped forceps.

FIGS. 5A-5B illustrate examples of cupped forceps.

FIG. 6 illustrates an embodiment of a cupped forceps having holes or slits.

FIG. 7 illustrates an embodiment of cupped forceps having a hook.

FIG. 8 illustrates an embodiment of a cupped forceps having a branched structure where more than one closed unit may be formed.

### DETAILED DESCRIPTION OF THE EMBODIMENTS

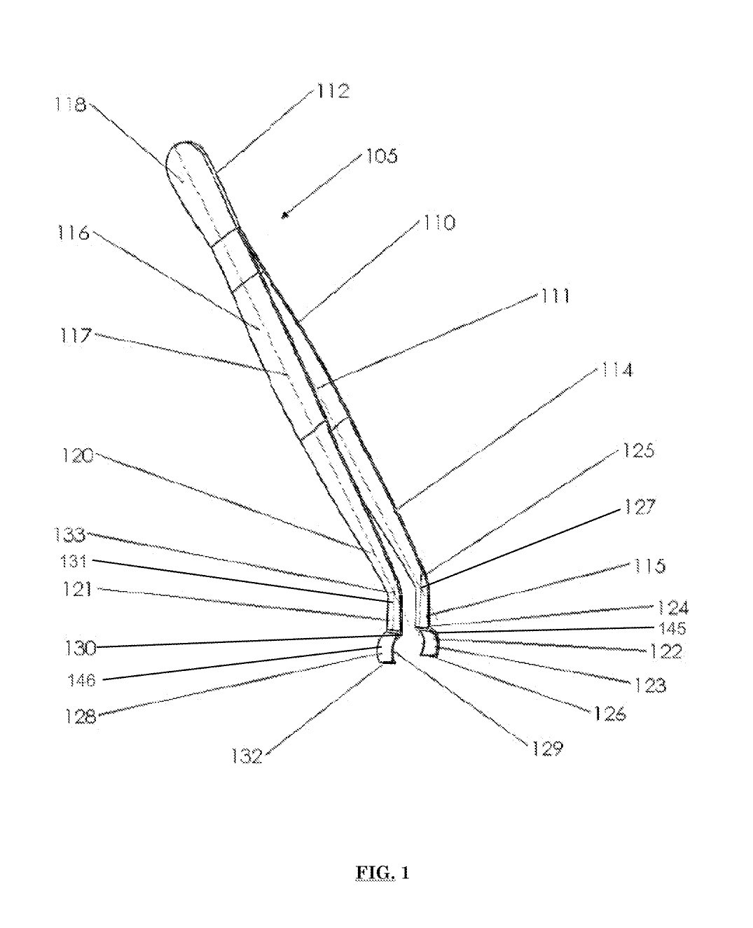

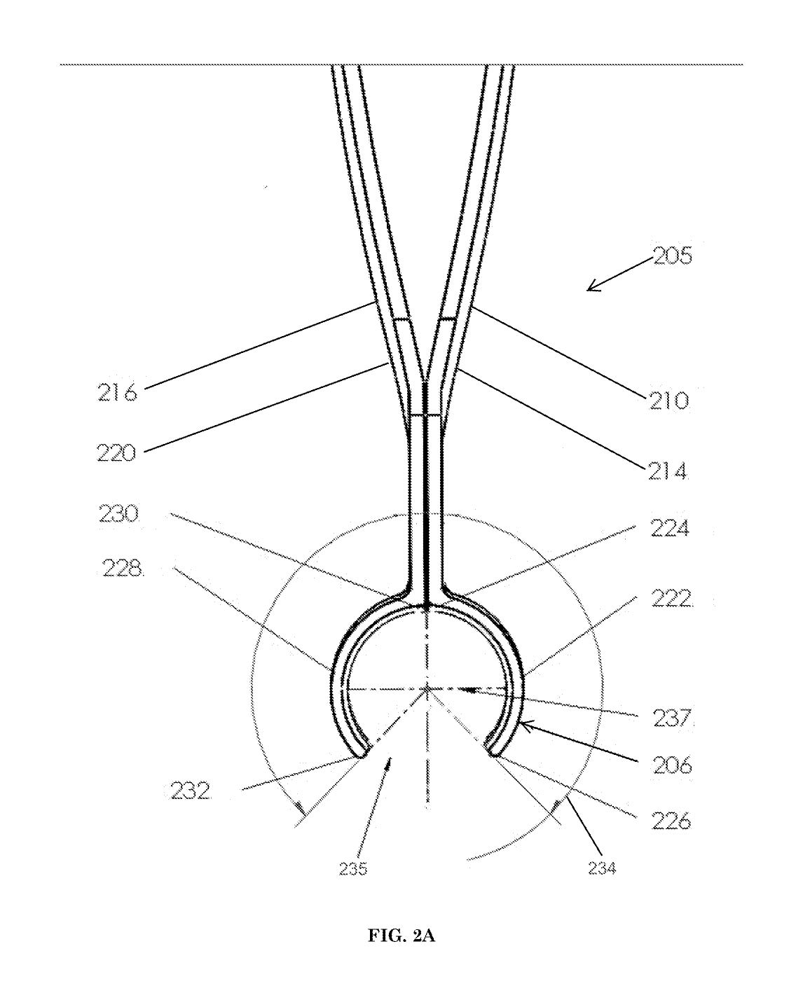

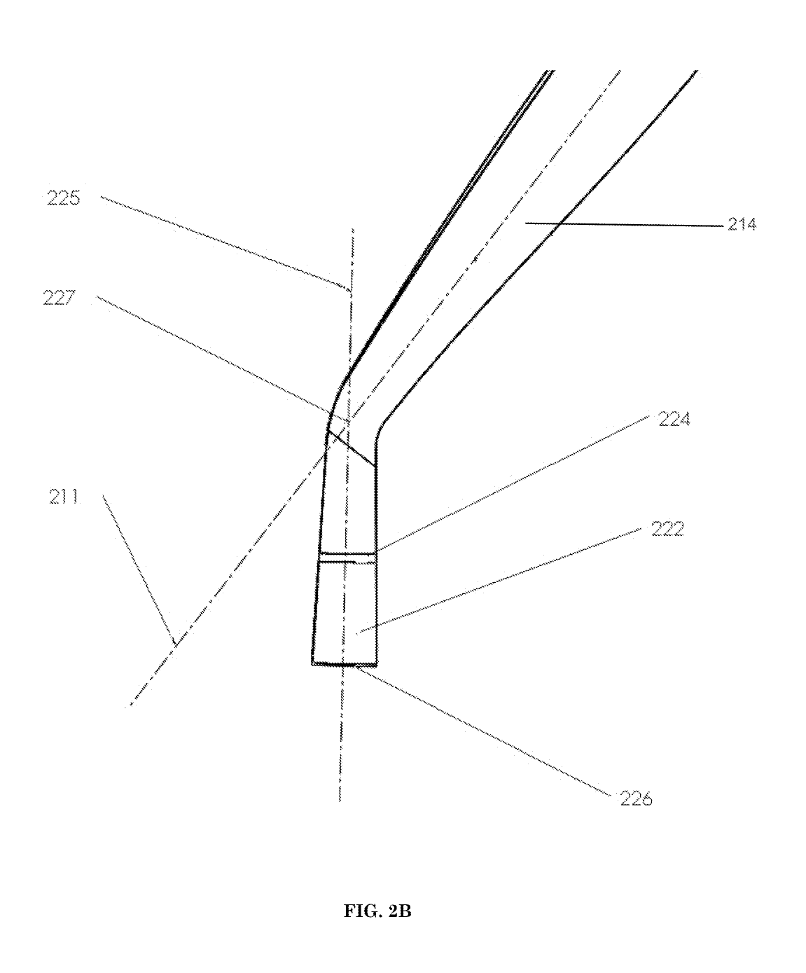

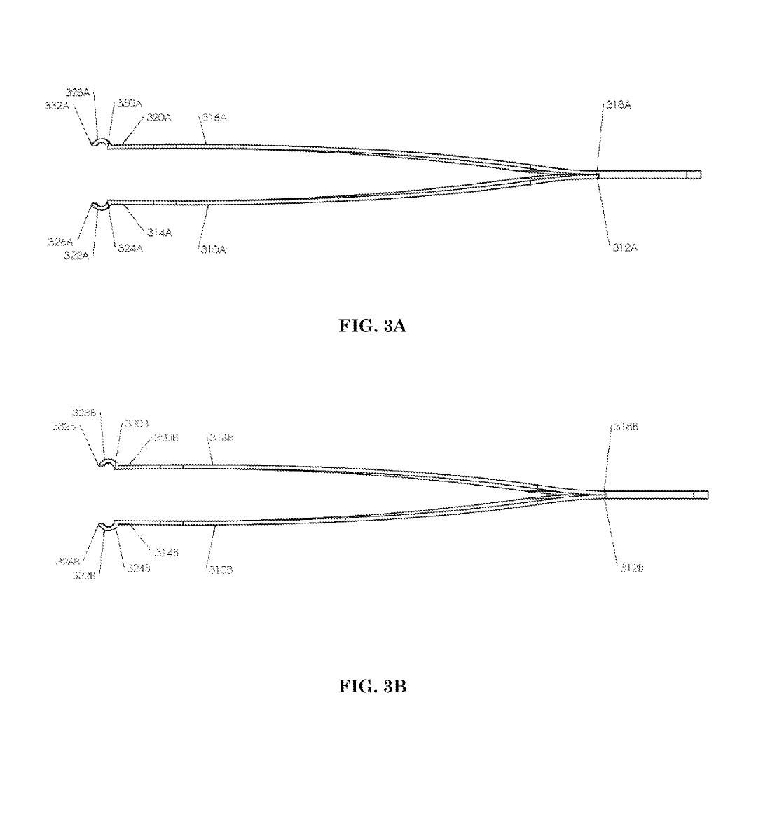

Certain terminology is used in the following description for convenience only and is not limiting. The words “right,” “left,” “top,” and “bottom” designate directions in the drawings to which reference is made. The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof. The FIGS illustrate embodiments of cupped forceps. Similar elements from FIG. to FIG. are numbered similarly. The elements in FIG. 1 are numbered in the one hundreds, and similar elements in FIGS. 2A and 2B are numbered in the two hundreds. Likewise, elements in FIGS. 3A-3B, 4A-4B, 5A-5C, 6, 7, and 8 that are similar to those in FIG. 1 are numbered in the three hundred, four hundreds, five hundreds, six hundreds, seven hundreds, and eight hundreds, respectively.

Referring to FIG. 1, an embodiment of a cupped forceps is illustrated. A cupped forceps may include a first shaft 110, with a first proximal end 114 and a first distal end 112, and a second shaft 116 with a second proximal end 120 and a second distal end 118. A first longitudinal axis 111 extends along the first shaft 110 from the first proximal end 114 to the first distal end 112. A second longitudinal axis 117 extends along the second shaft 116 from the second proximal end 120 to the second distal end 118. The first distal end 112 of the first shaft 110 may be connected to the second distal end 118 of the second shaft 116. The connection may be an integral connection or through bonding of the distal end 112 of the first shaft 110 to the distal end 118 of the second shaft 116. The bonding may be through a coupling, welding, attachment via an intermediate structure(s) fixed to the distal end 112 of the first shaft 110 and to the distal end 118 of the second shaft 116, or any other suitable bonding known to the skilled artisan.

A first cupped tip 122 may have a first concavity 123 between a first tip end 124 and a second tip end 126, and may be connected at the first proximal end 114 of the first shaft 110 through the first tip end 124. The connection of the first tip end 124 to the first proximal end may be direct, or as illustrated in FIG. 1 through a first proximal bend 115. A second cupped tip 128 may have a second concavity 129, a third tip end 130, and a fourth tip end 132, and may be connected at the second proximal end 120 of the second shaft 116 through the third tip end 130. The connection of the third tip end 130 to the second proximal end may be direct, or as illustrated in FIG. 1 through a second proximal bend 121. The connection of the first cupped tip 122 and the second cupped tip 128 to the respective proximal ends may be through a coupling, welding, integral, attachment via an intermediate structure(s) fixed to the respective proximal end 114, 120 and the respective cupped tip 122, 128, or any other suitable connection known to the skilled artisan. The connection illustrated in FIG. 1 is integral and through the intermediate structures of the proximal bend 115 and the proximal bend 121. The first cupped tip concavity 123 may face the second cupped tip concavity 129. The first cupped tip 122 may have a first cupped tip longitudinal axis 125 intersecting the first longitudinal axis 111 of the first shaft 110 at a first intersecting angle 127. The second cupped tip 128 may have a second cupped tip longitudinal axis 131 intersecting the second longitudinal axis 117 of the second shaft 116 at a second intersecting angle 133. In the embodiment illustrated in FIG. 1, the first cupped tip longitudinal axis is shown on the first proximal bend 115 and the second cupped tip longitudinal axis is shown on the second proximal end 121. As shown in FIG. 2B, the proximal ends and the cupped tips align to have a common longitudinal axis. The first interesting angle 127 and the second intersecting angle 133 may be any angle. The first intersecting angle 127 may be selected independently in comparison to the second intersecting angle 133 and vice versa. The first intersecting angle 127 and the second intersecting angle 133 may be identical or nearly identical to one another.

The first intersection angle and the second intersecting angle may be configured to allow convenient orientation of an object. For example, the angles may be configured in a manner that the cupped forceps have the ability to hold a scaffold in a secure position and orientation for trimming of the scaffold to ideal size to match a lesion cavity. The first intersecting angle and the second intersecting angle may be any angle in a range between any two integer values from 0° to 90°. In an embodiment, the first intersecting angle is 75° and the second intersecting angle is 75°. In an embodiment, the first intersecting angle is 15° and the second intersecting angle is 15°. In an embodiment, the first intersecting angle and the second intersecting angle are 45°±2°. The first intersecting angle and the second intersecting angle may be an angle in range between any two integer angle values from 15° to 52°, or ±2° of the angle.

The first cupped tip 122 and the second cupped tip 128 are described above and illustrated in FIG. 1 as connected to the respective first proximal end 114 and the second proximal end 120 through the respective proximal ends associated with the respective first tip end 124 and third tip end 130. However, embodiments may include one or both of the first cupped tip 122 or the second cupped tip 128 connected to its respective proximal end 114, 120 or an intermediate structure at any other point on the cupped tip. For example, the first proximal end or an intermediate structure may be connected to the back 145 of the first cupped tip 122 and the second proximal end or an intermediate structure may be connected to the back 146 of the second cupped tip 128. The connection point between a proximal end and a cupped tip may be independently selected for each of the first cupped tip 122 and the second cupped tip 128.

The first shaft 110 and the second shaft 116 may be pivotable toward each other about a pivot axis to a closed position. The pivot point may be at or near the joining point where the first distal end 112 of the first shaft 110 is connected to the second distal end 118 of the second shaft 116. The first shaft 110 and the second shaft 116 may be pivotable away from each other about the pivot axis to an open position. The first tip end 124 may meet the third tip end 130, and the second tip end 126 may be advanced toward the fourth tip end 132 in the closed position to form a closed unit. FIG. 2A illustrates a cupped forceps 205 with a closed unit 206.

In the closed unit, the first cupped tip longitudinal axis and the second cupped tip longitudinal axis may be parallel to one another.

The closed unit may be a configuration where the second tip end and the fourth tip end contact one another. This may be referred to as a “360° circumference” to indicate the contact between the second tip end and fourth tip end, and that the inner circumference of the first cupped tip and the second cupped tip are contiguous at the second tip end and the fourth tip end in the closed unit. The inner circumference may also be contiguous at the first tip end and the third tip end in the closed unit. However, the term “circumference” is not meant to limit the shape of the cupped tips to partial circles, or the closed unit in the 360° circumference configuration to a circle. The shapes of the cupped tips may be partial circles, but may also be other suitable shapes configured such that when the second tip end and the fourth tip end are advanced toward one another, a space remains between the first cupped tip and the second cupped tip to accommodate an object. In embodiments where the first and second cupped tips form a circle in the closed position, the “360° circumference” refers to a circle formed when the second tip end and the fourth tip end contact on another, and the first tip end and the third tip end contact one another. The 360° circumference closed unit may be in the configuration of a cylinder. The 360° circumference closed unit may be in the configuration of a cone. The skilled artisan will appreciate the coordination between geometries of the cupped tips to form a cone. The wider end of the cone may be selected from either side of the closed unit. Configurations other than a cylinder or cone are possible in embodiments herein and the configuration of the tip ends and cupped tips necessary to achieve any one configuration will be apparent to the skilled artisan.

Referring to FIG. 2A, an embodiment of a closed unit of a cupped forceps is illustrated with a first shaft 210 with a first proximal end 214, and a second shaft 216 with second proximal end 220. A first cupped tip 222 with a first tip end 224 and a second tip end 226 is connected to the proximal end 214, and a second cupped tip 228 with third tip end 230 and fourth tip end 232 is connected to the second proximal end 220. In the embodiment illustrated in FIG. 2A, the closed unit is in a configuration where the second tip end 226 and the fourth tip end 232 do not contact one another in the closed unit and a gap 235 spans between the second tip end 226 and the fourth tip end 232. The configuration where the second 226 and fourth 232 tip ends do not contact one another in the closed unit may be described as a closed unit having a circumference less than 360°. The circumference 234 illustrated in FIG. 2A shows a less than 360° circumference of 270°. As with the 360° circumference, the term “circumference” applied to the less than 360° circumference configuration is not meant to limit the shape of the cupped tips to partial circles. The shapes of the cupped tips may be partial circles, but may also be other suitable shapes configured such that when the second and fourth tip ends are advanced toward one another, a space remains between the first cupped tip and the second cupped tip to accommodate an object. The less than 360° circumference may be any circumference less that 360°. The less than 360° circumference may be any circumference less that 360° but greater than 180°. The less than 360° circumference may be selected from integer values from 180° to 359°. The less than 360° circumference may in a range between any two integer values selected from 180° to 359°. The less than 360° circumference may be 270°±15°, 270°±2°, or 270°. As with the 360° circumference embodiment, the closed unit in the less than 360° circumference embodiment may include a cylindrical, conical, or other shape as described above but with the gap remaining between the second and fourth tip ends. The skilled artisan will recognize the configurations of the tip ends and cupped tips to achieve any one configuration.

As described above, the cupped tips are configured such that when the second and fourth tip ends are advanced toward one another, a space remains between the first cupped tip and the second cupped tip to accommodate an object. The object accommodated between the first cupped tip and the second cupped tip may be any shape. The object may be cylindrical. The object may be a collection of two or more structures. The object may be easily crushed material, and the cupped forceps may be utilized to handle the easily crushed material with reduced or eliminates hazard of crushing in comparison to handling with traditional forceps. For example, the cupped forceps may be utilized to handle a fragile and easily crushed cylindrical object. The object may be neural implant. The object may be a scaffold. The object may be a medical device, including but not limited to a scaffold, a nerve conduit, a vascular graft, a tendon graft, or a stent. The cylindrical object may also be tissue, including but not limited to spinal cord, nerve, tendon, artery, vein, or bone. The cylindrical object may be any of the above in combination with an implantable wrap or sheath material surrounding the object. Examples of implantable wrap or sheath material can be found in U.S. patent application Ser. Nos. 12/567,589 (Pritchard et al., filed Sep. 25, 2009) and 13/692,458 (Langer et al., filed Dec. 3, 2012), and International Application No. PCT/US2012/046687 (Harvey et al., filed Jul. 13, 2012), which are incorporated herein by reference as if fully set forth.

All of the parts of the cupped forceps may be an integral unit. Two or more parts of a cupped forceps may be integrally formed with one another. Parts of a cupped forceps may be separately formed and joined together by any suitable joining. The two parts of the cupped forceps may be integrally joined by welding, glue, epoxy, solvent bonding, melting, soldering, brazing, nut and bolt, or other hardware fastener means.

Again referring to FIG. 2A, the closed unit may be configured in an open-ended cylindrical shape. The closed unit may include any closed diameter 237. For example, the closed unit may have an inner, closed diameter from 1 mm to 15 mm, or an inner, closed diameter in a range between any two integer diameters from 1 mm to 15 mm. As a further example, the closed diameter may be 1.0 mm±0.5 mm, 2.0 mm±0.5 mm, 3.0 mm±0.5 mm, 4.0 mm±1 mm, 6.0 mm±1 mm, 8.0 mm±1 mm, 10.0 mm±1 mm, 12.0 mm±1 mm, or 15.0 mm±1.5 mm. The closed unit may include any length from front to back (in FIG. 2A front is the side of the closed unit viewed, and the back is the opposite side). For example, the closed unit may include a length from 1 mm to 75 mm, or be in a range between any two integer values selected from 1 mm to 75 mm. As a further example, the closed unit may be a length of 2.0 mm±1 mm, 4.0 mm±1 mm, 6.0 mm±2 mm, 10.0 mm±3 mm, 15.0 mm±5 mm, 20.0 mm±5 mm, 30.0 mm±10 mm, or 50.0 mm±25 mm. Cupped forceps may be used to grip a polymer scaffold used as a biomedical implant. In an embodiment, the closed unit is designed to match a scaffold diameter such that compressive forces are spread across the scaffold surface to allow implantation without scaffold deformation. The closed unit may have a closed inner circumference from 180 to 360° to allow a scaffold to be trimmed, while being supported on a flat surface. Where the closed inner circumference is less than 360°, the scaffold maybe positioned such that a portion of it is contacted and supported by a surface near the closed unit during trimming, or any other manipulation of the scaffold. In addition, the closed unit may define a minimal diameter, which may be prevent scaffold deformation when the scaffold is closed, or “clamped,” in the closed unit.

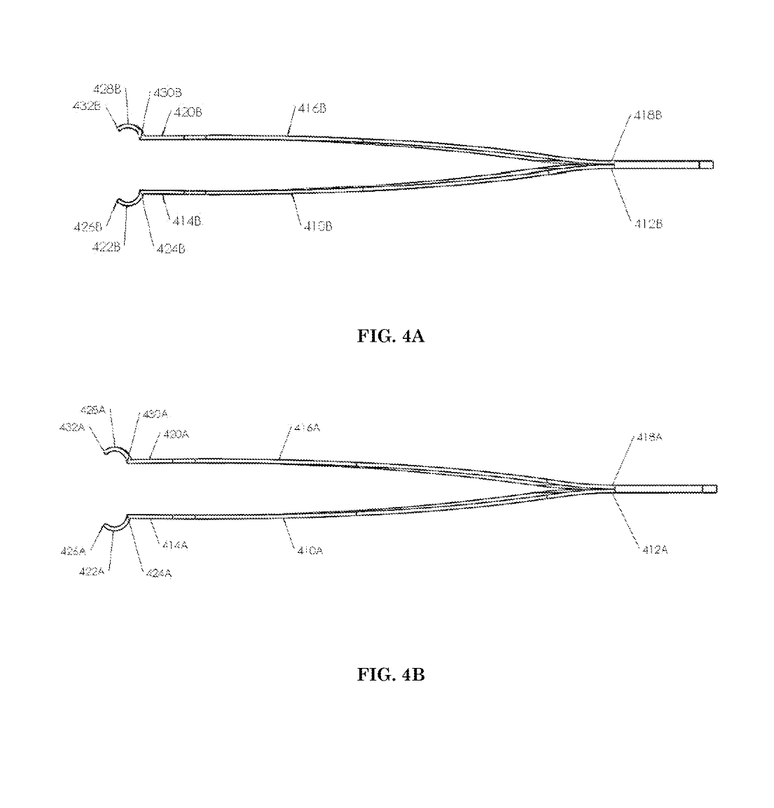

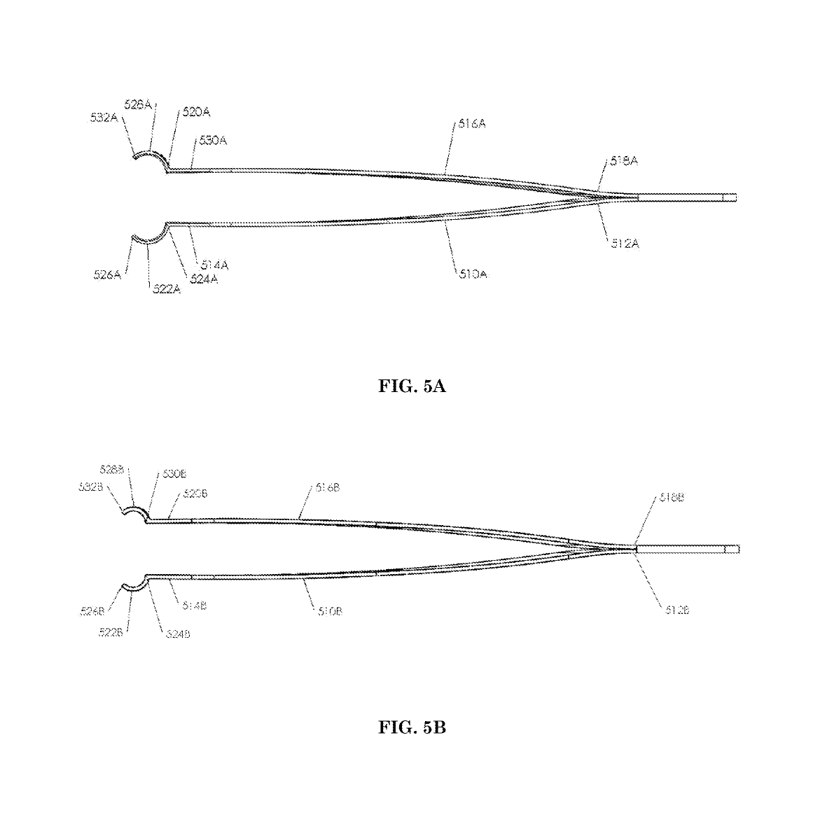

FIG. 3A illustrates an example of a cupped forceps with a closed unit diameter of 1 mm, and FIG. 3B illustrates an example cupped forceps with a closed unit diameter of 1.5 mm. FIG. 4A illustrates an example of a cupped forceps with a closed unit diameter of 2 mm, and FIG. 4B illustrates an example of a cupped forceps with a closed unit diameter of 3 mm. FIG. 5A illustrates an example of a cupped forceps with a closed unit diameter of 6 mm, and FIG. 5B an example of a cupped forceps with a closed unit diameter of 4 mm.

In the biased open position, the open distance between the back of the first cupped tip and the second cupped tip may be any suitable open distance. Suitability may be determined by the end use of the cupped forceps as described herein. The open distance may be adapted to allow passage of the tips around an object without extra outward force to avoid contact with the object. The open distance may be but is not limited to 1.3 cm, 1.1 cm, 1.5 cm, or 1.2 cm. The open distance may be any distance in a range between any to integer value distances selected from 1 to 3 cm.

The length from front to back of a closed unit may be any suitable distance. Suitability may be determined by the end use of the cupped forceps as described herein. The length of a closed unit may be 0.4 cm or 3.8 mm. The length of the closed unit may be a length in a range between any two integer value mm increments from 1 mm to 1 cm.

The bend distance extending from a) the junction between a proximal end and a proximal bend to b) the extreme tip end (for example tip end 126) may be any suitable distance. Suitability may be determined by the end use of the cupped forceps as described herein. The bend distance may be but is not limited to 1.4 cm. The bend distance may be in a range between any two integer value mm increments from 1 mm to 5 cm.

In an embodiment, the combination of biased open position, length of closed unit, and bend distance may be 1.2 cm, 0.4 cm, and 1.4 cm, respectively. In another embodiment, the combination of biased open position, length of closed unit, and bend distance may be 1.3 cm, 0.4 cm, and 1.4 cm, respectively. In another embodiment, the combination of biased open position, length of closed unit, and bend distance may be 1.1 cm, 0.4 cm, and 1.4 cm, respectively. In another embodiment, the combination of biased open position, length of closed unit, and bend distance may be 1.5 cm, 3.8 mm, and 1.4 cm, respectively. Each of these embodiments may have a closed unit with an angle ranging between and including any two integer values from 180° to 360°. In an embodiment, the closed unit angle is 225°. In another embodiment the closed unit angle is 315°. In an embodiment, the closed unit angle is 270°±15°, 270°±2°, or 270°.

A cupped forceps may be any suitable length. Suitability may be determined by the end use of the cupped forceps as described herein. An exemplary, non-limiting length of a cupped forceps is 11.5 cm. A cupped forceps may have but is not limited to a length in a range between any to 0.5 cm increments from 2.5 to 25 cm.

The cupped forceps may include cupped tips at a length from front to back to allow accurate trimming of a cylindrical or tubular object within the cupped tips to a length equivalent to the length of the cupped tips. The cupped forceps may include markings, such as a ruler along one or both of the cupped tips, for accurate measurement. The edge of the cupped tips may be flush to allow for a blade to cut along the cupped tips and produce objects of a specific length.

In an embodiment, the first cupped tip and the second cupped tip are adjustable to change the size and shape of the closed unit. For example, the first cupped tip and the second cupped tip may increase in size to increase the size of the closed unit or the first cupped tip and the second cupped tip may decrease in size to decrease the size of the closed unit. The first cupped tip and the second cupped tip may be capable of changing shape in order to change the shape of the closed unit.

In an embodiment, the cupped forceps contain a mechanical stop to prevent further compression between the first cupped tip and the second cupped tip. The mechanical stop may be located between the first shaft and the second shaft. The mechanical stop may be located between the first cupped tip and the second cupped tip. The mechanical stop may be any compression stop. For example, the compression stop may be a pin, a collar, or a lock.

In an embodiment, the cupped forceps contain a closure to maintain the cupped forceps in the closed position. The closure may be any closure. For example, the closure may be a lock, a pin, a collar or clasp.

The cupped forceps may be made of any material. In an embodiment, the cupped forceps may be made of a material that can withstand sterilization techniques. For example, the cupped forceps may be made of stainless steel or plastic. In an embodiment, the cupped forceps may be made of a material or color that prevents glare from bright lights in an operating room setting. The cupped forceps may be made entirely or at least in part with a transparent plastic configured to visualize at least a portion of the object within the cupped tips. The transparent plastic may be but is not limited to polypropylene.

In an embodiment, at least one of the cupped tips and/or at least one of the cupped tip ends may be serrated to improve the grip of the object. Such serrations may include teeth and perforations.

Embodiments include methods of handling objects. The method may include enclosing an object in a cupped forceps. The cupped forceps may be any one described herein. Enclosing may refer to the object being within or partially within the closed unit. The object may be a fragile and easily crushed material. The object accommodated between the first cupped tip and the second cupped tip may be any shape. The object may be cylindrical. The object may be a collection of two or more structures. The object may be easily crushed material, and the cupped forceps may be utilized to handle the easily crushed material with reduced or eliminate hazard of crushing in comparison to handling with traditional forceps. For example, the cupped forceps may be utilized to handle a fragile and easily crushed cylindrical object. The object may be neural implant. The object may be a scaffold. The object may be a medical device, including but not limited to a scaffold, a nerve conduit, a vascular graft, a tendon graft, or a stent. Examples of medical devices can be found in U.S. patent application Ser. Nos. 12/567,589 (Pritchard et al., filed Sep. 25, 2009), 13/692,458 (Langer et al., filed Dec. 3, 2012), 61/779,429 (Reynolds et al., filed Mar. 13, 2013), 61/779,489 (Reynolds et al., filed Mar. 13, 2013), 61/839,179 (Prifti et al., Jun. 25, 2013), and International Application No. PCT/US2012/046687 (Harvey et al., Jul. 13, 2012), which are incorporated herein by reference as if fully set forth. The cylindrical object may also be tissue, including but not limited to spinal cord, nerve, tendon, artery, vein, or bone. The method may include manipulating the object while it is enclosed in the cupped forceps. The method may include placing a portion of the object exposed in the gap of a closed unit of a cupped forceps against a support during at least one of before, during, or after manipulating the object. Manipulating may include trimming the object. Trimming the object may be done to customize the object for its end use. For example, a medical device may be trimmed to fit the position in which it will be implanted. Manipulating may include exposing the object to different chemical environments. The exposing step may introduce new buffer or active agent components to the object, or replace components in which the object was stored with working buffer or active agents.

Embodiments of a method of handling an object include methods of manipulating scaffolds for use in the treatment of neural injury. The neural injury may be spinal cord injury. For example, the cupped forceps may be designed and utilized for scaffold handling and transport. Scaffolds, which may be made from resorbable polymer such as PLGA-PLL, are intended for use in the treatment of complete functional spinal cord injury—ASIA “A” classification to improve motor and sensory function. These scaffolds have a generally geometric cylindrical shape to best approximate the spinal cord and fit a range of lesion cavities in thoracic spinal cord injury. Due to the hydrated composition and porous material, the scaffolds require extreme care when handling. Embodiments of the method include a method of manipulating a scaffold for use in the treatment of spinal cord injury. The step of enclosing may include gripping the scaffold with any one of the cupped forceps contained herein. The step of manipulating may include revising the scaffold. Revising the scaffold may include trimming the scaffold. The method may include implanting the scaffold. Implanting the scaffold may include inserting the scaffold in a lesion cavity in a spinal cord injury. The spinal cord injury may be a thoracic spinal cord injury.

The cupped forceps in a method of handling an object may include cupped tips at a length to allow accurate trimming of a cylindrical or tubular object within the cupped tips to a length equivalent to the length of the cupped tips. The cupped forceps may include markings, such as a ruler along one or both of the cupped tips, for accurate measurement. The edge of the cupped forceps may be flush to allow for a blade to cut along the cupped tips and produce objects of a specific length. The step of manipulating may include trimming the object to a length equivalent to the length of the cupped tips. The step of manipulating may include trimming the object to a length measured by markings along one or both of the cupped tips. The step of manipulating may include cutting the object to length by sliding a blade along one or both ends of the closed unit.

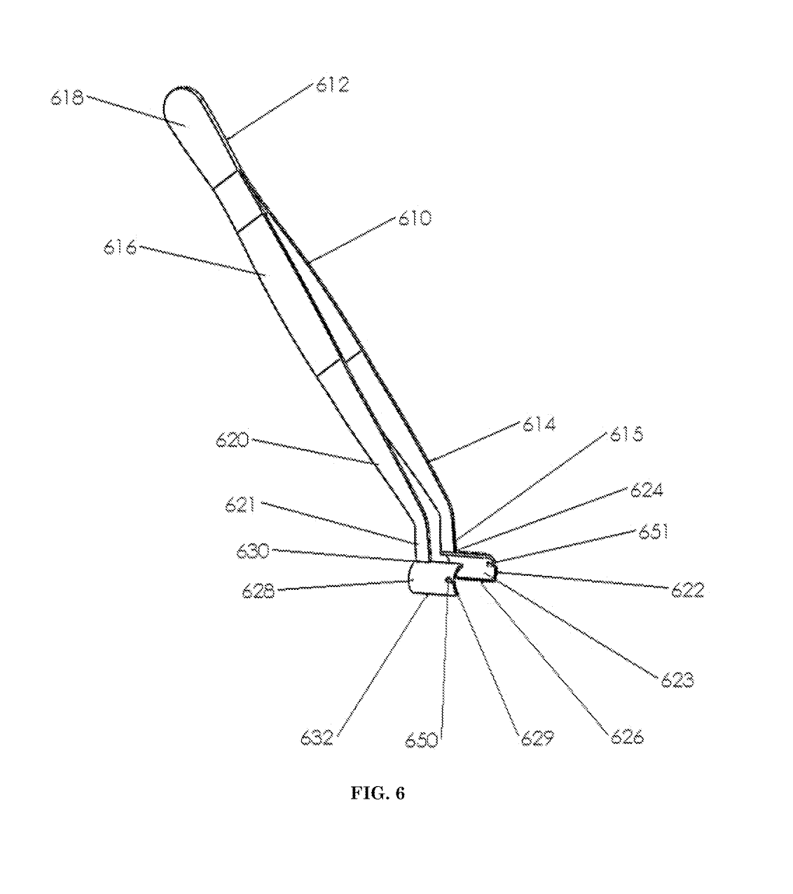

Embodiments of a method of handling objects include methods of manipulating conduits or wraps for use in the treatment of peripheral nerve injuries. For example, the cupped tip along one side of the cupped forceps may be extended in comparison to the other or the cupped tips may be of equal length. See FIG. 6, which illustrates another embodiment of a cupped forceps herein. The elements of FIG. 6 are numbered similar to those in FIG. 1, but with the addition of 650. The method may include grasping a nerve end with the extended cupped forceps, and placing a nerve conduit with the extended cupped forceps. The extended cupped tips 624, 628 could support the nerve end, as the conduit is slipped over the nerve end. The shafts of the forceps would act as a mechanical stop to allow nerve ends to be placed into conduits at a specific depth in the range of 1 mm to 5 mm, or any depth in a range selected from any two integers between 1 mm and 5 mm. The forceps could maintain the positioning of both the nerve and the conduit to assist in the attachment of the nerve end to the conduit. In one embodiment, the cupped forceps may have holes or slits 650, 651 on the edges of the tips to allow for ease of suturing or attachment of the conduit to the nerve end. The number of slits per tip could be 1 to 10 slits, or any number between and including 1 to 10 slits. The number of slits per tip could be 2 to 4 slits.

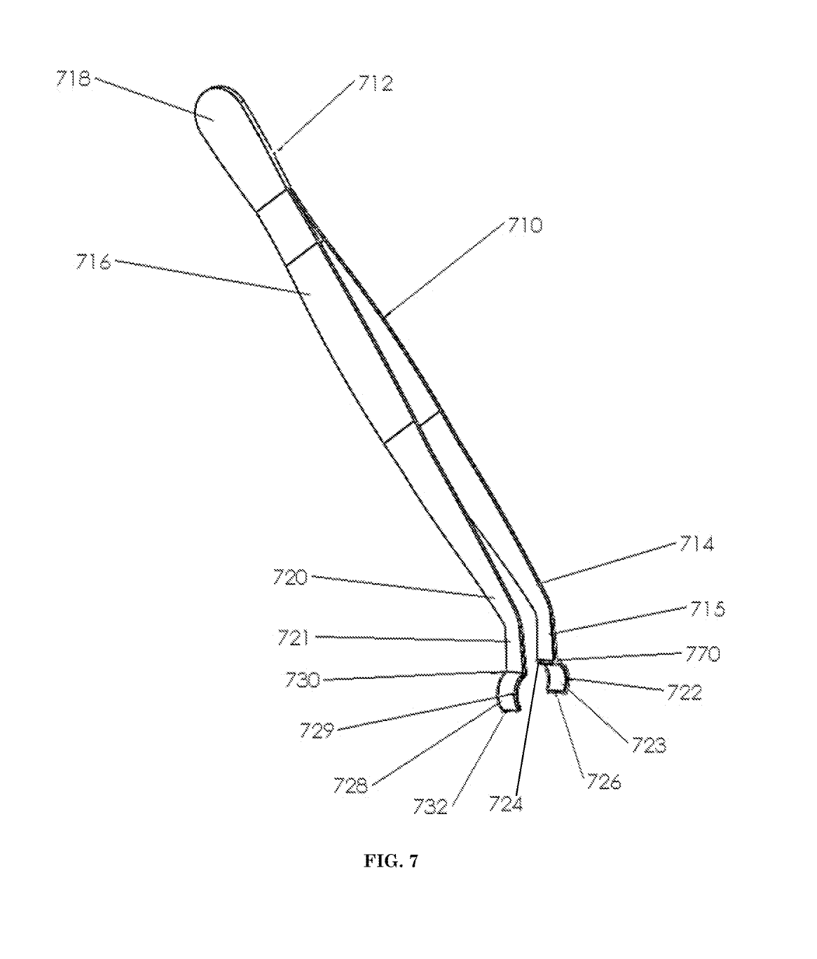

Referring to FIG. 7, an embodiment of the cupped forceps could include a hook 770 at the corner of a cupped tip. FIG. 7 illustrates the hook 770 at a specific corner of a specific cupped tip, but alternate embodiments may include one or more hooks at various positions. The hooked and cupped forceps could assist in the tubular formation of a nerve wrap around an injured nerve. The corner of the wrap could be pierced and held in place by the hook, while the second end is wrapped around the nerve. The hook on the cupped forceps could hold the wrap in place, as it is being sutured or attached to the nerve. The hook, could be released by either trimming the pierced location, or sliding the hook of the cupped forceps out of the piercing. A method of handling an object may include one or more of these steps while implementing a cupped forceps including a hook at the corner of a cupped tip. Features in FIG. 7 similar to those in FIG. 1 are numbered similarly, but with the addition of 600.

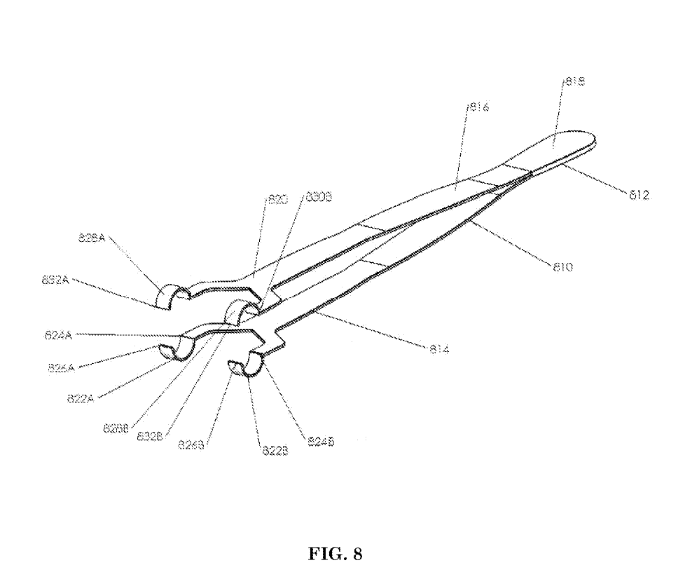

Referring to FIG. 8, an embodiment of the cupped forceps could be branched to have two or more pairs of cupped ends at varying distances apart to allow for grasping along a lengthy cylindrical or tubular object. The cupped tips may be connected to prongs extending from the branch point(s). For example, the branched cupped forceps could be used to hold a tissue, including a peripheral nerve, at a specific distance. The specific distance may be 1 mm to 20 cm apart. The specific distance may be a distance in a range between and including any two integers selected 1 mm to 20 cm apart. The branched cupped forceps would assist in maintaining distance and alignment of the tubular or cylindrical object, while the object is manipulated, including trimmed, sutured, and glued. The method of handling objects may include one or more of these steps and be implemented with a branched cupped forceps. Features in FIG. 8 similar to those in FIG. 1 are numbered similarly but with the addition of 700. Also, branched features are labeled “a” or “b” on their specific branch.

### Embodiments

The following list includes particular embodiments. The list, however, is not limiting and does not exclude the embodiments otherwise described herein or alternate embodiments.

1. A cupped forceps comprising:

a first shaft having a first proximal end and a first distal end;

a second shaft having a second proximal end and a second distal end;

the first distal end of the first shaft connected to the second distal end of the second shaft;

a first cupped tip having a first concavity between a first tip end and a second tip end, the first tip end connected at the first proximal end of the first shaft;

a second cupped tip having a second concavity between a third tip end and a fourth tip end, the third tip end connected at the second proximal end of the second shaft,

wherein the first cupped tip concavity faces the second cupped tip concavity, the first shaft and the second shaft are pivotable towards each other about a pivot axis to a closed position, and the first tip end is configured to meet the third tip end and the second tip end and the fourth tip end are configured to be advanced towards each other to form a closed unit in the closed position.

2. The cupped forceps of embodiment 1, wherein the first tip end is connected to the first proximal end of the first shaft through a first intermediate structure between the first tip end and first proximal end, and the third tip end is connected to the second proximal end of the second shaft through a second intermediate structure between the third tip end and the second proximal end.

3. The cupped forceps of embodiment 2, wherein the first intermediate structure is a first proximal bend and the second intermediate structure is a second proximal bend.

4. The cupped forceps of embodiment 3, wherein the first proximal end, the first proximal bend, and the first cupped tip are an integral unit; and the second proximal end, the second proximal bend, and the second cupped tip are an integral unit.

5. The cupped forceps of any one or more of embodiments 1-4, wherein the cupped forceps is an integral unit.

6. The cupped forceps of any one or more of embodiments 1-5, wherein the closed unit has a circumference of less than 360°.

7. The cupped forceps of embodiment 6, wherein the circumference is 270°±15°.

8. The cupped forceps of any one or more of embodiments 1-7, wherein the closed unit has an inner, closed diameter from 1 mm to 15 mm.

9. The cupped forceps of one or more of embodiments 1-8, wherein the closed unit has a length from 1 mm to 75 mm.

10. The cupped forceps of any one or more of embodiments 1-9, wherein the first cupped tip and the second cupped tip are adjustable to change the size and shape of the closed unit.

11. The cupped forceps of any one or more of embodiments 1-10, wherein the first cupped tip includes a first cupped tip longitudinal axis intersecting a first longitudinal axis of the first shaft at a first intersecting angle, and the second cupped tip includes a second cupped tip longitudinal axis intersecting a second longitudinal axis of the second shaft at a second intersecting angle, the first cupped tip longitudinal axis and the second cupped tip longitudinal axis being an angle of 45°±15°.

12. The cupped forceps of any one or more of embodiments 1-11 further comprising a mechanical stop between the first shaft and the second shaft to prevent further compression between the first cupped tip and the second cupped tip.

13. The cupped forceps of claim any one or more of embodiments 1-12 further comprising a closure to maintain the cupped forceps in the closed position.

14. The cupped forceps of any one or more of embodiments 1-13 further comprising at least one of: at least one slit on the first cupped tip and at least one slit on the second cupped tip.

15. The cupped forceps of any one or more of embodiments 1-14 further comprising a hook at a corner of at least one of the first cupped tip or the second cupped tip.

16. The cupped forceps of any one or more of embodiments 1-15 further comprising a first proximal bend between the first tip end and the first proximal end to form a first corner where the first proximal bend and the first tip end meet and a second proximal bend between the second tip end and the second proximal end to form a second corner where the second proximal bend and the second tip end meet, wherein the cupped forceps further comprises a hook on at least one of the first corner or the second corner.

17. The cupped forceps of any one or more of embodiments 1-16 further comprising a first branch at the first proximal end of the first shaft and a second branch at the second proximal end of the second shaft, wherein the first branch includes one or more first prongs connected to one or more additional first branch cupped tips and the second branch include one or more second prongs connected to one or more additional second branch cupped tips.

18. A cupped forceps comprising:

a first shaft having a first proximal end and a first distal end;

a second shaft having a second proximal end and a second distal end;

the first distal end of the first shaft connected to the second distal end of the second shaft;

a first branch having a first branch proximal end and a first branch distal end, the first branch distal end connected to the first proximal end of the first shaft, and the first branch proximal end having a first prong and a second prong;

a second branch having a second branch proximal end and a second branch distal end, the second branch distal end connected to the second proximal end of the second shaft, and the second branch proximal end having a third prong and a fourth prong;

a first cupped tip having a first concavity between a first tip end and a second tip end, the first tip end connected at the first prong;

a second cupped tip having a second concavity between a third tip end and a fourth tip end, the third tip end connected at the third prong,

a third cupped tip having a third concavity between a fifth tip end and a sixth tip end, the fifth tip end connected at the second prong;

a fourth cupped tip having a fourth concavity between a seventh tip end and an eighth tip end, the seventh tip end connected at the fourth prong,

wherein the first cupped tip concavity faces the second cupped tip concavity, the third cupped tip concavity faces the fourth cupped tip concavity, the first shaft and the second shaft are pivotable towards each other about a pivot axis to a closed position, and the first tip end is configured to meet the third tip end, the fifth tip end is configured to meet the seventh tip end, the second tip end and the fourth tip end are configured to be advanced towards each other to form a closed unit in the closed position, and the sixth tip end and the eighth tip end are configured to be advanced towards each other to form the closed unit in the closed position.

19. Any cupped forceps contained herein.

20. A method of handling an object comprising:

enclosing the object in a cupped forceps, wherein the cupped forceps comprises any cupped forceps of any one or more of embodiments 1-19.

21. A method of handling an object comprising:

enclosing the object in a cupped forceps, wherein the cupped forceps comprises:

a first shaft having a first proximal end and a first distal end;

a second shaft having a second proximal end and a second distal end;

the first distal end of the first shaft connected to the second distal end of the second shaft;

a first cupped tip having a first concavity between a first tip end and a second tip end, the first tip end connected at the first proximal end of the first shaft;

a second cupped tip having a second concavity between a third tip end and a fourth tip end, the third tip end connected at the second proximal end of the second shaft,

wherein the first cupped tip concavity faces the second cupped tip concavity, the first shaft and the second shaft are pivotable towards each other about a pivot axis to a closed position; and the first tip end is configured to meet the third tip end, and the second tip end and the fourth tip end are configured to be advanced toward each other to form a closed unit in the closed position; and enclosing includes pivoting the first shaft and the second shaft toward each other.

22. The method of embodiment 21, wherein the pivoting is performed until the closed unit is formed in the closed position.

23. The method of any one or more of embodiments 20-22 further comprising manipulating the object.

24. The method of embodiment 23, wherein manipulating includes trimming the object.

25. The method of embodiment 24, wherein manipulating includes exposing the object to a chemical environment.

26. The method of any one or more of embodiments 20-25, wherein the object is a tissue.

27. The method of embodiment 26, wherein the object is a spinal cord, a nerve, a tendon, an artery, a vein, or a bone.

28. The method of any one or more of embodiments 20-27, wherein the object further comprises an implantable wrap or sheath material surrounding the object.

29. The method of any one or more of embodiments 20-25 and 28, wherein the object is a medical device.

30. The method of embodiment 29, wherein the medical device is a scaffold, a nerve conduit, a vascular graft, a tendon graft, or a stent.

31. The method of embodiment 29, wherein the medical device is a scaffold.

32. The method of embodiment 31, wherein the step of enclosing includes enclosing the scaffold within the closed unit.

33. The method of any one or more of embodiments 31-32 further comprising manipulating the scaffold for use in the treatment of neural injury.

34. The method of embodiment 33, wherein the neural injury is a spinal cord injury.

35. The method of any one or more of embodiments 31-34, wherein manipulating includes revising the scaffold.

36. The method of embodiment 35, wherein revising includes trimming the scaffold.

37. The method of any one or more of embodiments 31-36 further comprising implanting the scaffold.

38. The method of embodiment 37, wherein implanting the scaffold includes inserting the scaffold in a lesion cavity in a spinal cord injury.

39. The method of embodiment 38, wherein the spinal cord injury is a thoracic spinal cord injury.

40. Any method of handling an object contained herein.

Further embodiments herein may be formed by supplementing an embodiment with one or more element from any one or more other embodiment herein, and/or substituting one or more element from one embodiment with one or more element from one or more other embodiment herein.

It is understood, therefore, that this invention is not limited to the particular embodiments disclosed, but is intended to cover all modifications which are within the spirit and scope of the invention as defined by the appended claims; the above description; and/or shown in the attached drawings.

What is claimed is:

1. A cupped forceps comprising:
a first shaft having a first proximal end and a first distal end;
a second shaft having a second proximal end and a second distal end;
the first distal end of the first shaft connected to the second distal end of the second shaft;
a first cupped tip having a first concavity between a first tip end and a second tip end, the first tip end connected at the first proximal end of the first shaft;
a second cupped tip having a second concavity between a third tip end and a fourth tip end, the third tip end connected at the second proximal end of the second shaft,
wherein the first cupped tip concavity faces the second cupped tip concavity, the first shaft and the second shaft are pivotable towards each other about a pivot axis to a closed position; and the first tip end is configured to meet the third tip end and the second tip end and the fourth tip end are configured to be advanced toward each other to form a closed unit in the closed position.

2. The cupped forceps of claim 1, wherein the first tip end is connected to the first proximal end of the first shaft through a first intermediate structure between the first tip end and the first proximal end, and the third tip end is connected to the second proximal end of the second shaft through a second intermediate structure between the third tip end and the second proximal end.

3. The cupped forceps of claim 2, wherein the first intermediate structure is a first proximal bend and the second intermediate structure is a second proximal bend.

4. The cupped forceps of claim 3, wherein the first proximal end, the first proximal bend, and the first cupped tip are an integral unit, and the second proximal end, the second proximal bend, and the second cupped tip are an integral unit.

5. The cupped forceps of claim 1, wherein the cupped forceps is an integral unit.

6. The cupped forceps of claim 1, wherein the closed unit has a circumference of less than 360°.

7. The cupped forceps of claim 6, wherein the circumference is 270°±15°.

8. The cupped forceps of claim 1, wherein the closed unit has an inner, closed diameter from 1 mm to 15 mm.

9. The cupped forceps of claim 1, wherein the closed unit has a length from 1 mm to 75 mm.

10. The cupped forceps of claim 1, wherein the first cupped tip and the second cupped tip are adjustable to change the size and shape of the closed unit.

11. The cupped forceps of claim 1, wherein the first cupped tip include a first cupped tip longitudinal axis intersecting a first longitudinal axis of the first shaft at a first intersecting angle, and the second cupped tip includes a second cupped tip longitudinal axis intersecting a second longitudinal axis of the second shaft at a second intersecting angle, the first cupped tip longitudinal axis and the second cupped tip longitudinal axis being an angle of 45°±15°.

12. The cupped forceps of claim 1 further comprising a mechanical stop between the first shaft and the second shaft to prevent further compression between the first cupped tip and the second cupped tip.

13. The cupped forceps of claim 1 further comprising a closure to maintain the cupped forceps in the closed position.

14. The cupped forceps of claim 1 further comprising at least one of: at least one slit on the first cupped tip and at least one slit on the second cupped tip.

15. The cupped forceps of claim 1 further comprising a hook at a corner of at least one of the first cupped tip or the second cupped tip.

16. The cupped forceps of claim 1 further comprising a first proximal bend between the first tip end and the first proximal end to form a first corner where the first proximal bend and the first tip end meet and a second proximal bend between the second tip end and the second proximal end to form a second corner where the second proximal bend and the second tip end meet, wherein the cupped forceps further comprises a hook on at least one of the first corner or the second corner.

17. The cupped forceps of claim 1 further comprising a first branch at the first proximal end of the first shaft and a second branch at the second proximal end of the second shaft, wherein the first branch includes one or more first prongs connected to one or more additional first branch cupped tips and the second branch includes one or more second prongs connected to one or more additional second branch cupped tips.

18. A cupped forceps comprising:
a first shaft having a first proximal end and a first distal end;
a second shaft having a second proximal end and a second distal end;
the first distal end of the first shaft connected to the second distal end of the second shaft;
a first branch having a first branch proximal end and a first branch distal end, the first branch distal end connected to the first proximal end of the first shaft, and the first branch proximal end having a first prong and a second prong;
a second branch having a second branch proximal end and a second branch distal end, the second branch distal end connected to the second proximal end of the second shaft, and the second branch proximal end having a third prong and a fourth prong;
a first cupped tip having a first concavity between a first tip end and a second tip end, the first tip end connected at the first prong;
a second cupped tip having a second concavity between a third tip end and a fourth tip end, the third tip end connected at the third prong,
a third cupped tip having a third concavity between a fifth tip end and a sixth tip end, the fifth tip end connected at the second prong;
a fourth cupped tip having a fourth concavity between a seventh tip end and an eighth tip end, the seventh tip end connected at the fourth prong,
wherein the first cupped tip concavity faces the second cupped tip concavity, the third cupped tip concavity faces the fourth cupped tip concavity, the first shaft and the second shaft are pivotable towards each other about a pivot axis to a closed position, and the first tip end is configured to meet the third tip end, the fifth tip end is configured to meet the seventh tip end, the second tip end and the fourth tip end are configured to be advanced towards each other to form a closed unit in the closed position, and the sixth tip end and the eighth tip end are configured to be advanced towards each other to form the closed unit in the closed position.

19. A method of handling an object comprising:
enclosing the object in a cupped forceps, wherein the cupped forceps comprises:
a first shaft having a first proximal end and a first distal end;
a second shaft having a second proximal end and a second distal end;
the first distal end of the first shaft connected to the second distal end of the second shaft;
a first cupped tip having a first concavity between a first tip end and a second tip end, the first tip end connected at the first proximal end of the first shaft;
a second cupped tip having a second concavity between a third tip end and a fourth tip end, the third tip end connected at the second proximal end of the second shaft,
wherein the first cupped tip concavity faces the second cupped tip concavity, the first shaft and the second shaft are pivotable towards each other about a pivot axis to a closed position, and the first tip end is configured to meet the third tip end and the second tip end and the fourth tip end are configured to be advanced toward each other to form a closed unit in the closed position, and enclosing includes pivoting the first shaft and the second shaft toward each other.

20. The method of claim 19 further comprising manipulating the object.

21. The method of claim 20, wherein manipulating includes trimming the object.

22. The method of claim 20, wherein manipulating includes exposing the object to a chemical environment.

23. The method of claim 19, wherein the object is a tissue.

24. The method of claim 23, wherein the object is a spinal cord, a nerve, a tendon, an artery, a vein, or a bone.

25. The method of claim 19, wherein the object further comprises an implantable wrap or sheath material surrounding the object.

26. The method of claim 19, wherein the object is a medical device.

27. The method of claim 26, wherein the medical device is a scaffold, a nerve conduit, a vascular graft, a tendon graft, or a stent.

28. The method of claim 26, wherein the medical device is a scaffold.

29. The method of claim 28, wherein the step of enclosing includes enclosing the scaffold within the closed unit.

30. The method of claim 28 further comprising manipulating the scaffold for use in the treatment of neural injury.

31. The method of claim 30, wherein the neural injury is a spinal cord injury.

32. The method of claim 30, wherein manipulating includes revising the scaffold.

33. The method of claim 32, wherein revising includes trimming the scaffold.

34. The method of claim 30 further comprising implanting the scaffold.

35. The method of claim 34, wherein implanting the scaffold includes inserting the scaffold in a lesion cavity in a spinal cord injury.

36. The method of claim 35, wherein the spinal cord injury is a thoracic spinal cord injury.
